(19) United States
(12) Patent Application Publication
(10) Pub. No.: US 2015/0157872 A1
(43) Pub. Date: Jun. 11, 2015

(54) Device for Treating Cancer by Hyperthermia and the Method Thereof

(71) Applicants: Bhaskar Vishwanathan, Attibele (IN); Nagadeva K. Singh, (US); EFD Induction Private Limited, Attibele (IN)

(72) Inventors: Bhaskar Vishwanathan, Bangalore (IN); Nagadeva K. Singh, Bangalore (IN)

(73) Assignee: EFD Induction Private Limited, Attibele (IN)

(21) Appl. No.: 14/562,669

(22) Filed: Dec. 6, 2014

(30) Foreign Application Priority Data
Dec. 6, 2013 (IN) 5615/CHE/2013

(51) Int. Cl. A61N 2/00; A61K 41/00; A61N 2/02

(52) U.S. Cl. CPC A61N 2/004; A61N 2/02; A61K 41/0052; A61N 2/002

(57) ABSTRACT

The present invention provides a device for treating tumor and cancer in humans and animals by using hyperthermia. The device is customizable according to size of the tumor as well as the location i.e. specific body part that is affected by disease. The device generates magnetic induction that in turn heats the magnetic nanoparticle and kills the cancerous cell without affecting the neighboring cells.

### DESCRIPTION OF THE INVENTION

The following specification particularly describes the invention and the manner in which it is to be performed:

### TECHNICAL FIELD OF THE INVENTION

The present invention relates to the use of a device for treating cancer by hyperthermia, more specifically to the use of induction coil that applies electromagnetic fields to magnetic nanoparticles injected in the affected area and treats diseased cells and tissues such as tumors and cancers.

### BACKGROUND OF THE INVENTION

Cancer is a class of diseases, characterized by out of control cell growth. There are different types of cancers and each is classified by the type of cell that is initially affected. Cancer is ultimately a result of cells that grow uncontrollably and do not die. Normal cells in the body follow an orderly path of growth, division, and death. Programmed cell death is called apoptosis and when this process is deregulated, cancer is initiated. Unlike regular cells, cancer cells do not experience programmed cell death and instead continue to grow and divide which leads to a mass of abnormal cells that grow out of control. There are different types of cancers such as breast cancer, skin cancer, lung cancer, colon cancer, prostate cancer, lymphoma etc.

Presently, hyperthermia is delivered by various techniques like microwave, ultrasound, radio frequency and for deep seated tumors, by interstitial arrays. These methodologies have their own limitations, for example, they heat up the surrounding normal tissues when heat is conducted from external source to reach the tumor within the body and are invasive when needles are inserted for interstitial hyperthermia. These draw backs could be avoided by using magnetic nanoparticles injected within the tumor and later applying induction hyperthermia.

U.S. Pat. No. 7,588,565B2 discloses a device for use in anastomosis of tissue comprising a biocompatible material and a means of applying radio frequency energy or electrical energy to generate heat within the said biocompatible material. The device may also be used to bond or fuse at two materials where at least one of the material is a tissue. It also discloses methods to anastomose tissue or to bond or to fuse these materials using these devices. There is a need of a device that is flexible to be used in different kinds of patients and can be used for treating humans and animals afflicted with a disease like tumor or cancer.

In another US Application No. US20070196281A1 discloses unique treatment methods and innovative articles that can be placed in a human or animal body to enable controlled destruction of diseased tissue. The methods include destruction of diseased cells and tissues by magnetically controlled motion and an externally controllable drug delivery process with a capability to start and stop the drug delivery at any time, for any duration. This technology does not provide a device that can be customized according to need of different patients or devices that can be placed in close proximity with the diseased part of patient's body and provides enough heat and medication that can kill the cancerous cell without affecting neighboring cells.

Hence looking at the state of the art technology, there is a need of a device and a method to treat tumor and cancer in humans as well as animals. It is desired to use a device that provides electromagnetic radiations externally to magnetic nanoparticle which in turn delivers the curing agents to the diseased site and destroy cancerous cells by heat produced due to magnetic induction combined with chemotherapeutic as well as by immunologic and radio therapeutic and genetic means.

### SUMMARY OF THE INVENTION

The present invention provides a device used for treating cancer by hyperthermia concomitantly with chemo-radio therapeutic and monoclonal antibody, genetic application. The device comprises an induction coil, an insulation layer to cover the induction coil in order to protect an individual from electric shock, an external layer covering the insulation layer to provide comfort for the individual when placed in proximity of the tumor and a power supply given to the induction coil to generate magnetic field depending on size of the tumor.

A method of killing tumor formed in any body parts of humans and animals by hyperthermia and chemo radio therapeutic modalities. The method starts with injecting magnetic nanoparticles/conjugated with multifunctional nanoparticles(chemo, immuno, radio therapeutic) in the tumor, then placing the device in close proximity of the tumor, hence applying magnetic field to the nanoparticles that provides heat as well as tumoricidal effects and destroy the cancerous cells.

The device is customizable according to size of the tumor as well as the location i.e. specific body part that is affected by disease. The device generates magnetic induction that in turn heats the magnetic nanoparticle and kills the cancerous cell without affecting the neighboring cells.

### BRIEF DESCRIPTION OF DRAWINGS

The features of embodiments will become more apparent from the following detailed description of embodiments when read in conjunction with the accompanying drawings. Furthermore, elements that are known to be common and well understood to those in the industry are not depicted in order to provide a clear view of the various embodiments of the invention. Thus, in the interest of clarity and conciseness, the drawings are generalized in form.

FIG. 1 illustrates the structural features of the device in accordance with an embodiment of the invention.

FIG. 2 illustrates the use of the device in treating readily accessible tumors on the body surface in accordance with an embodiment of the invention.

FIG. 3 illustrates the use of the device for treating cervical tumors and vaginal tumors in accordance with an embodiment of the invention.

FIG. 4 illustrates the use of the device for treating tumor on the face in accordance with an embodiment of the invention.

FIG. 5 illustrates the use of the device for treating tumor on the neck in accordance with an embodiment of the invention.

FIG. 6 illustrates the positioning of the device for treating tumor in human body in accordance with an embodiment of the invention.

### DETAILED DESCRIPTION OF THE INVENTION

Unless otherwise defined, all technical and scientific terms used herein have the meaning commonly understood by a person skilled in the art to which this invention belongs. One skilled in the art will recognize many methods and materials similar or equivalent to those described herein, which could be used in the practice of this invention. Indeed, this invention is in no way limited to the methods and materials described.

In order to more clearly and concisely describe and point out the subject matter of the claimed invention, the following definitions are provided for specific terms which are used in the following written description.

By the term “Cancer” we mean a class of diseases characterized by out-of-control cell growth, as context requires.

By the term “Chemotherapeutic, radio therapeutic, immunotherapeutic, and gene modifying agents” or “Multifunctional nanoparticles” we mean a combination of specific chemical, gene modifying agents or drugs with the magnetic nanoparticle that are compactly packaged in a singular entity but are selectively destructive in their individual capacities to malignant cells and tissues for treatment of cancer, as context requires.

By the term “induction coil” we mean a type of inductor used to produce high frequency electromagnetic pulses.

By the term “nanoparticle” we mean an electron microscopy particle with at least one dimension less than 100 nm, as context requires.

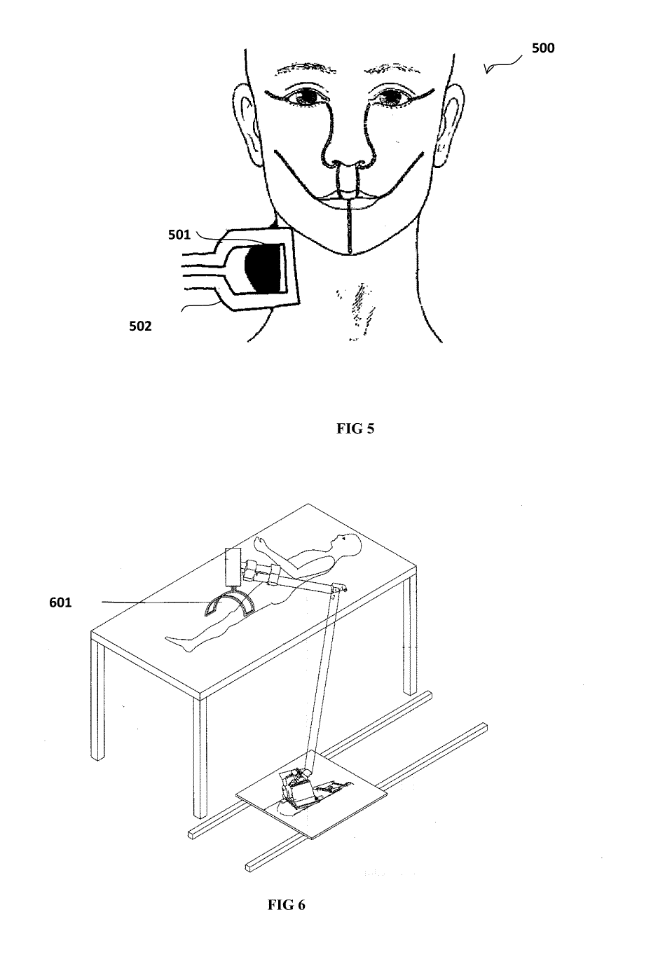

The present invention relates to a device used for treating cancers and tumor. The device is a combination of flexible induction coil and insulating cover, which is individualized for different cancer patients, taking into consideration the site and size of the tumor. This coil is placed in contact with the tumor after injecting it with magnetic nanoparticles. FIG. 6 shows the positioning of the device (601) on certain body part where the tumor has been formed. The magnetic nanoparticles are manually injected within the tumor to obtain the best homogenous distribution as feasible. Later the tumor is heated with the device (601).

The carrier used in the magnetic device is iron oxide nanoparticles, chemotherapeutic agents such as cisplatin, doxorubicin, mitomycin, methotrexate, or temozolomide, gene modifying agents, monoclonal antibodies for tumor antigens or for antiangiogenesis, hormonal receptors and radio nucleotides. The magnetic material is used as encapsulated structures or in the form of small particles such as nano magnetic materials. The magnetic nanoparticles have to be combined with drugs and targeting biomolecules or radio therapeutic radio nucleotides in the form of biocompatible suspension for diagnostic and therapeutic applications. Magnetic fluids are the dispersion of single domain particles of nanometre size less than 30 nm in carrier liquids. Generally aqueous carrier liquids are used taking into consideration biocompatibility issues.

Magnetite (Fe₃O₄) may be used for hyperthermia. Magnetic fluids having ferromagnetic or super paramagnetic particles in aqueous suspension are used for hyperthermia, the procedure is called magnetic fluid hyperthermia. It is determined by specific absorption rate, which describes the amount of energy converted into heat per time and mass. Curie temperature is an important application where temperature dispersion is tailored, beyond which ferromagnetic materials lose their magnetic properties. It is also an important physical parameter to optimise the required temperature for the tumor.

Biocompatibility of magnetic fluid is also an important issue for successful magnetic fluid hyperthermia treatment. Here magnetic liposomes i.e. liposomes having encapsulated magnetic fluid have become popular for formulation of biocompatible magnetic fluid for biological application. Magnetic liposomes made of biocompatible phospholipids are used for magnetic fluid hyperthermia. Liposomes are used as a vehicle for the delivery of drugs to tumors. Solid liposomes having modified surface with polyethylene glycol is used. These liposomes are known to circulate in blood stream and passively accumulate in the tumour tissues due to leaky vessels of the tumour. For site specific triggered release, thermo sensitive and pH sensitive liposomes, micro gel or polymers are of great importance. Fusogenic liposomes may also be used. Site specific delivery is achieved by coating liposomes with ligands or antibodies that target overexpressed receptors in the tumour tissue. Cancer specific binding agents could include hormones, antibodies and viruses and radio nucleotides. Hormone receptors are used in breast, and prostate cancers. Monoclonal antibodies are used in head and neck cancers, brain tumours and lung tumours.

The device (100) is used for treating cancer by hyperthermia concomitantly with chemo-radio therapeutic, gene modifying agent and monoclonal antibody application and is customized according to the site and size of the tumor to be treated.

FIG. 1 shows flexible device which is a water cooled copper cable coil (102) externally covered with silica rubber (101), (104 and 105) are the end connectors of the device.

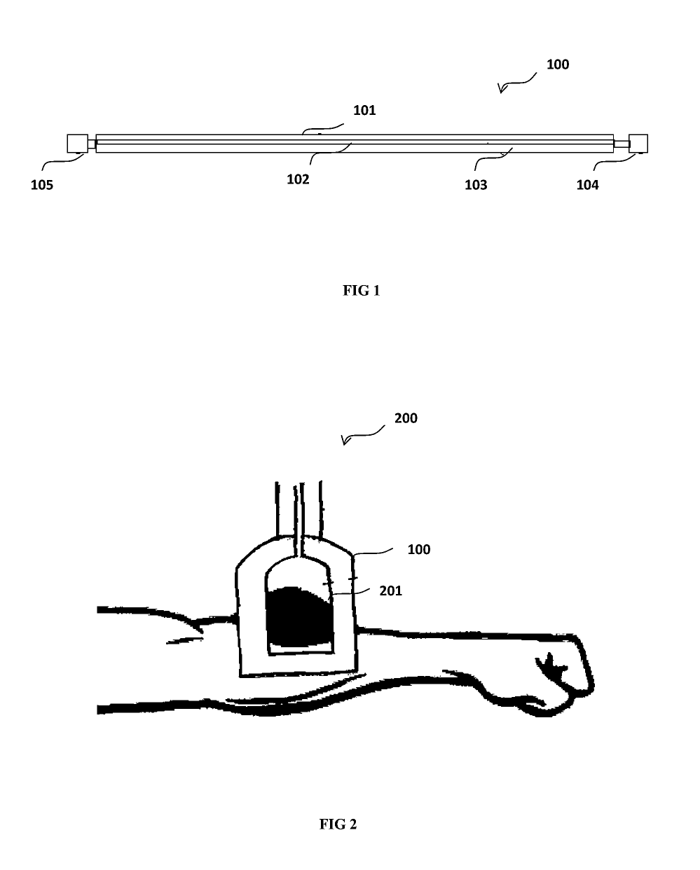

FIG. 2 shows the use of a rigid device to treat tumor readily accessible on the body surface where device (202) is placed at a site to target the tumor and kill the diseased cells using induction hyperthermia.

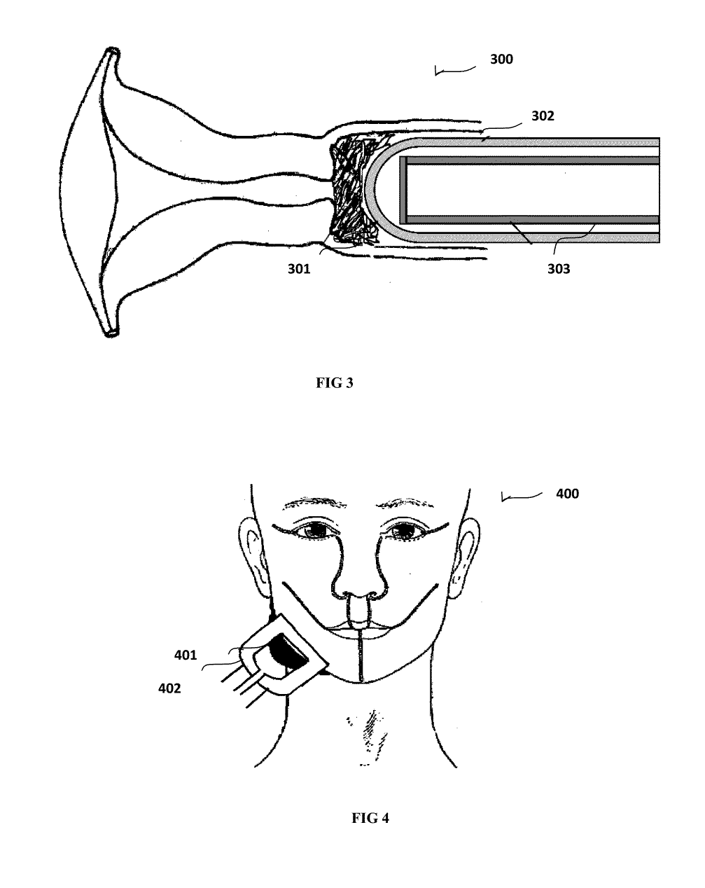

FIG. 3 shows the use of the device (100) in its rigid form to treat tumor formed in cervix of an individual. The tumor (301) is formed at a specific site in the cervix that is treated by induction hyperthermia using a device (100) that consists of an insulation layer (302) that is a plasma coating and an induction coil (303) that provides heat to the magnetic nanoparticles that is inserted in the tumor. FIG. 4 and FIG. 5 shows the use of the device to treat tumor formed at specific site in the face and neck.

The present invention provides a device that treats disease such as tumor and cancer in humans and animals. The device is customizable according to size of the tumor as well as the location i.e. specific body part that is affected by disease. The device generates magnetic induction that in turn heats the magnetic nanoparticle and kills the cancerous cell without affecting the neighboring cells.

We claim:

1. A device for treating cancer by hyperthermia, the device comprising:
a) an induction coil;
b) an insulation layer to cover the induction coil in order to protect an individual from electric shock;
c) an external layer covering the insulation layer to provide comfort for the individual when placed in proximity to the tumor;
d) a power supply connected to the induction coil to generate magnetic field depending on size of the tumor.

2. The device as claimed in claim 1 wherein said external layer is made of silica rubber.

3. The device as claimed in claim 1 wherein said induction coil is made of flexible copper cables.

4. The device as claimed in claim 1 wherein said induction coil is individualized for treating tumor of different size at various body parts.

5. The device as claimed in claim 1 wherein said insulation layer is made of plasma coating.

6. The device as claimed in claim 1 wherein the power supply ranges from 5 kW to 15 kW depending on the size of the tumor.

7. A method of destroying tumor formed inside the body of humans and animals by using hyperthermia, said method comprising the steps of:
a) injecting magnetic nanoparticles into the tumor;
b) applying magnetic field to the nanoparticles that provides heat and tumoricidal effects which destroys the tumor or cancerous cells.

8. The method as claimed in claim 7 wherein said nanoparticles consist of chemotherapeutic and radio therapeutic agents and monoclonal antibody.

9. The method as claimed in claim 7 wherein said nanoparticle is injected into the tumor using a porous needle.
